(19) United States
(12) Patent Application Publication
(10) Pub. No.: US 2021/0346593 A1
(43) Pub. Date: Nov. 11, 2021

(54) ORAL-HYDRATION SYSTEM, METHOD, AND KIT

(71) Applicants: Kurt Elliot Friedman, Plantation, FL (US); Russell Perry Kotch, Weston, FL (US)

(72) Inventors: Kurt Elliot Friedman, Plantation, FL (US); Russell Perry Kotch, Weston, FL (US)

(21) Appl. No.: 16/870,516

(22) Filed: May 8, 2020

(51) Int. Cl. A61M 3/02; A61F 5/56

(52) U.S. Cl. CPC A61M 3/0254; A61M 3/0279; A61M 2205/3334; A61M 2210/0625; A61F 2005/563; A61F 5/56

(57) ABSTRACT

An oral-hydration system may include a dispenser, and a port carried by the dispenser and the port enables a fluid under positive-pressure to be dispersed within the patient's mouth from the dispenser. The system may also include a positioner that positions the dispenser adjacent a premolar, adjoining a cheek, and away from an incisor.

### BACKGROUND

The embodiments relate to the field of oral health.

Compromised saliva production is a problem for some people for a host of varying reasons. The person with compromised salvia production can suffer affects to a number of their biological functions and/or a degrading of their quality of life.

### SUMMARY

According to an embodiment, an oral-hydration system may include a dispenser, and a port carried by the dispenser and the port enables a fluid under positive-pressure to be dispersed within the patient's mouth from the dispenser. The system may also include a positioner that positions the dispenser adjacent a premolar, adjoining a cheek, and away from an incisor.

The system may include further an interface that connects the dispenser to a pump through fluid communication. The system may additionally include an adjustable regulator that meters the fluid delivery to the patient.

The positioner may serve as a retainer, a denture, and/or a bruxism-guard. The port may be on the cheek side of the patient's mouth, point towards the roof of the patient's mouth, and/or the number of ports depend on the patient's requirements.

The port may be on the cheek side of the patient's mouth, point towards the bottom of the patient's mouth, and/or the number of ports depend on the patient's requirements. The port may be on the cheek side of the patient's mouth, point towards the roof and bottom of the patient's mouth, and/or the number of ports depends on the patient's requirements.

The system may also include a fitting carried by the dispenser that joins the interface to the pump. The positioner may be a custom molded design and/or a universal design.

The interface may be 2-6 mm surgical tubing. The dispenser may be 10-40 mm in length.

The positioner may be inserted by the patient, removed by the patient, and/or does not require dental anchors. The fluid may be water and/or distilled water.

Another aspect is a method for treating a patient with water where the patient is suffering from dry mouth, and the method may include setting the flowrate of water in an oral-hydration system at 3-7 milliliters per hour. The method may also include inserting the oral-hydration system in the patient's mouth for 4-9 hours. The method may further include removing the oral-hydration system from the patient's mouth after treatment.

The method may additionally include designing the oral-hydration system based upon the patient's physiology. The method may also include determining that the patient needs additional oral help, and adding a medication to the water to improve the patient's oral-hydration and/or oral-health. The fluid is at least one of water, distilled water, fluoride, and/or anti-microbial solution.

Another aspect is a kit for use in performing an oral-hydration procedure, and the kit includes a dispenser, and a port carried by the dispenser and the port enables a fluid under positive-pressure to be dispersed within the patient's mouth from the dispenser. The kit may further include a positioner positions the dispenser in contact with a premolar, adjoining a cheek, and away from an incisor. The kit may additionally include a pump, and an interface that connects the dispenser to the pump through fluid communication.

The kit may also include instructions that include dosing parameters including a flow rate through the dispenser, and the timing of the flow rate through the dispenser.

The kit may additionally include instructions that include setting the flowrate of the fluid in an oral-hydration system at 3-7 milliliters per hour, and inserting the oral-hydration system in the patient's mouth for 4-9 hours.

### BRIEF DESCRIPTION OF THE DRAWINGS

FIG. 1 is a schematic block diagram of a system in accordance with the embodiments.

FIG. 2 is a first exemplary representation of the system of FIG. 1.

FIG. 3 is a second exemplary representation of the system of FIG. 1.

FIG. 4 is a third exemplary representation of the system of FIG. 1.

FIG. 5 is a fourth exemplary representation of the system of FIG. 1.

FIG. 6 is a fifth exemplary representation of the system of FIG. 1.

FIG. 7 is a sixth exemplary representation of the system of FIG. 1.

FIG. 8 is a seventh exemplary representation of the system of FIG. 1.

FIG. 9 is an eighth exemplary representation of the system of FIG. 1.

FIG. 10 is a ninth exemplary representation of the system of FIG. 1.

FIG. 11 is a tenth exemplary representation of the system of FIG. 1.

FIG. 12 is a flowchart illustrating method aspects according to various embodiments.

FIG. 13 is a flowchart illustrating method aspects according to the method of FIG. 12.

FIG. 14 is a flowchart illustrating method aspects according to the method of FIG. 12.

### DETAILED DESCRIPTION

Embodiments will now be described more fully hereinafter with reference to the accompanying drawings, in which preferred embodiments are shown. Like numbers refer to like elements throughout.

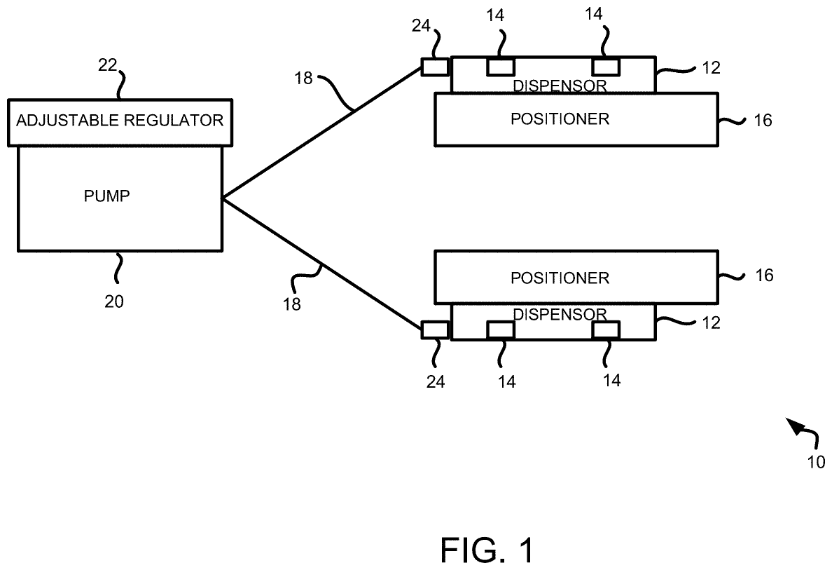

With reference now to FIG. 1, an oral-hydration system 10 is initially described. In one embodiment, the system 10 includes a dispenser 12, and a port 14 carried by the dispenser and the port enables a fluid (not shown) under positive-pressure to be dispersed within the patient's mouth from the dispenser. For example the fluid is distilled water. The system 10 also includes a positioner 16 that positions the dispenser 12 adjacent a premolar, adjoining a cheek, and away from an incisor of the patient (not shown). For instance, the positioner 16 holds the dispenser 12 in the described orientation within the patient's mouth during use.

In one embodiment, the system 10 further includes an interface 18 that connects the dispenser 12 to a pump 20 through fluid communication. In other words, the pump 20 drives the fluid, e.g. distilled water, to and through the dispenser 12 and out of port 14 through positive-pressure delivery. In one embodiment, the pump 20 is a medical grade pump.

In another embodiment, the system 10 additionally includes an adjustable regulator 22 that meters the fluid delivery to the patient. For instance, the adjustable regulator 22 varies the fluid flow delivered to the port 14 as selected by the patient. An alternative embodiment enables a medical practitioner to select and set the adjustable regulator 22 at given flow rate through port 14.

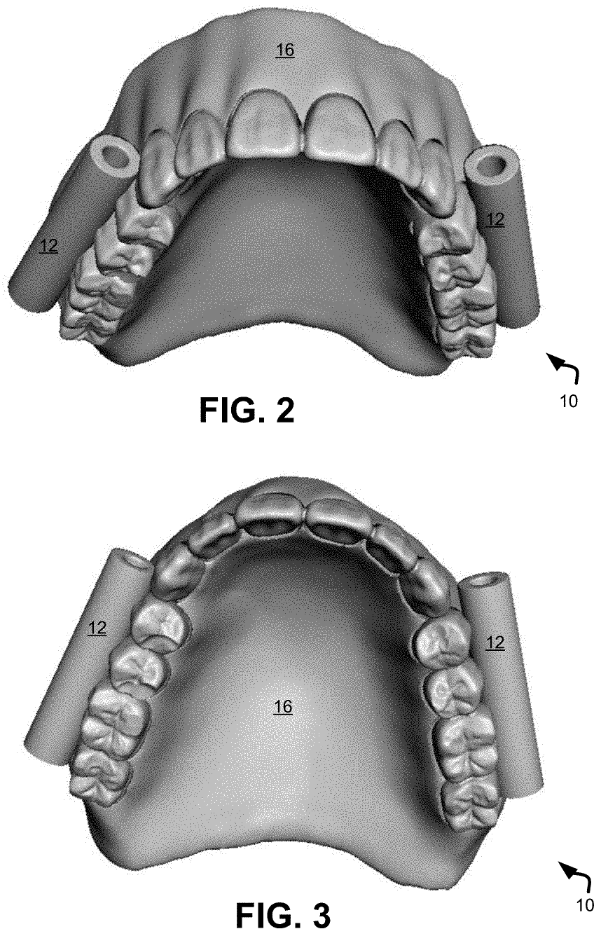

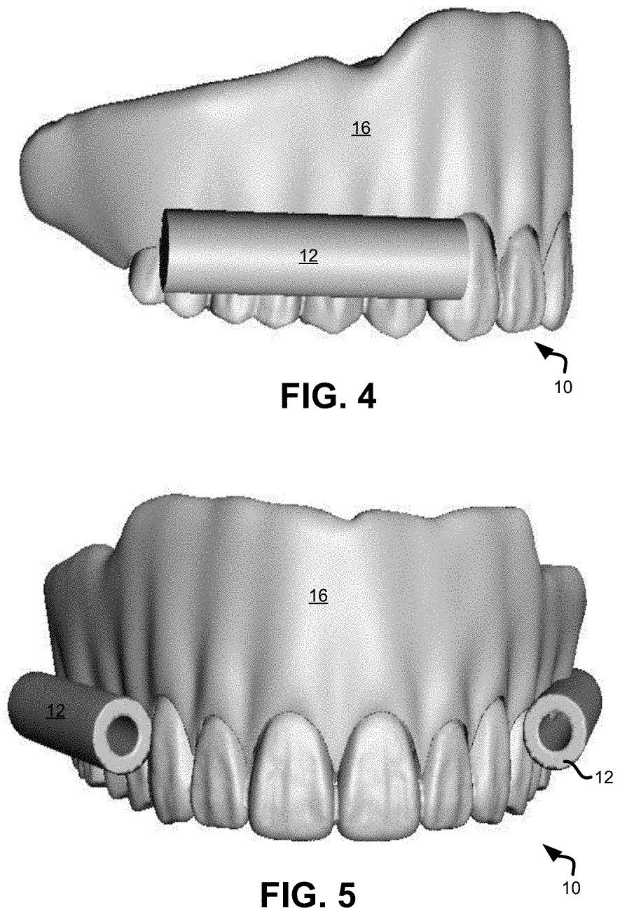

In one embodiment, the positioner 16 serves as a retainer, a denture, and/or a bruxism-guard. Stated another way, the positioner is designed and structurally fabricated to meet the requirements of a retainer, a denture, and/or a bruxism-guard while also delivering fluid through the port 14 for the patient. For example, with additional reference to FIGS. 2-5, a denture embodiment is illustrated. However, the system 10 does not provide impact protection to the teeth of the patient such as that provided by a sports mouth guard.

In another embodiment, the port 14 is on the cheek side of the patient's mouth and point towards the roof of the patient's mouth. In other words, the port 14 is aimed in the described directions by its placement on the dispenser 12 and by being located within the patient's mouth by the positioner 16. In one embodiment, the number of ports 14 depends on the patient's requirements such as to help regulate the flow rate of the fluid within the patient's mouth as well as to compensate for physical limitations present in the patient's mouth that present challenges for a standard layout of ports.

In another embodiment, the port 14 is on the cheek side of the patient's mouth, point towards the bottom of the patient's mouth, and/or the number of ports depend on the patient's requirements. In another embodiment, the port 14 is on the cheek side of the patient's mouth, point towards the roof and bottom of the patient's mouth, and/or the number of ports depends on the patient's requirements.

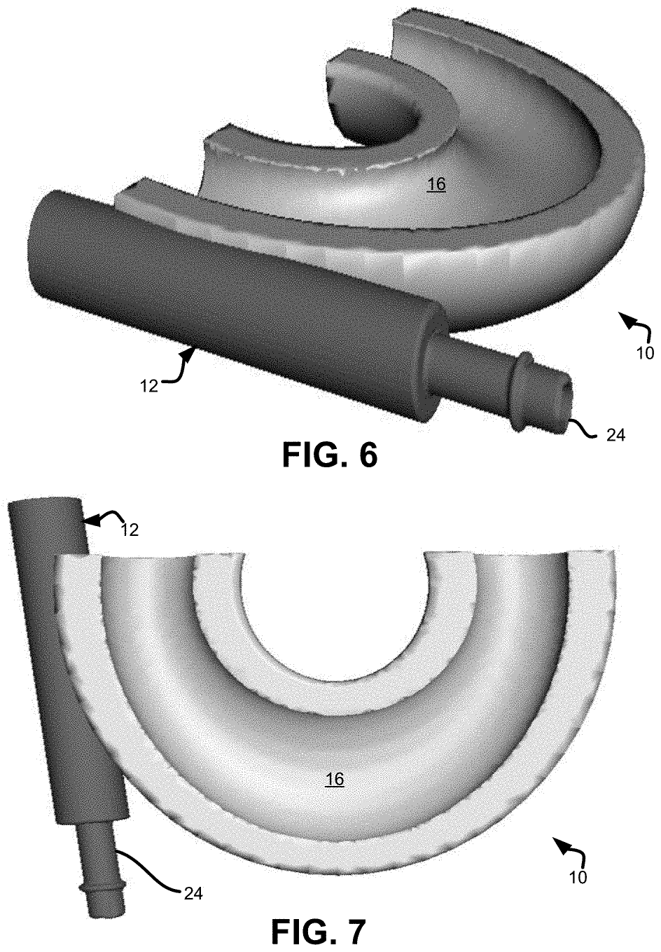

In one embodiment, with additional reference to FIGS. 6-7, the system 10 also includes a fitting 24 carried by the dispenser 12 that joins the interface 18 to the pump 20. For example, the fitting 24 is a nipple that the interface 18 is secured to. An alternative example is the fitting 24 is an opening in dispenser 12 that is sized to receive and secure the interface 18 through an interference fit. Additionally the fitting 24 permits fluid communication from the pump 20 to through the dispenser 12 via the interface 18.

In one embodiment, the positioner 16 is a custom molded design that accounts and compensates for a patient's individual physiology and medical needs. In another embodiment, the positioner 16 is a universal design that matches the physiology and medical needs of a patient as illustrated in FIGS. 6-7. In other words, the positioner 16 is designed to fit the most common patient mouth configuration/needs. In another embodiment, the positioner 16 is a universal design that matches the physiology and medical needs of a particular class of patients, e.g. small mouth, medium mouth, large mouth, some dry mouth, moderate dry mouth, excessive dry mouth, etc. In yet another embodiment, the positioner 16 is a combination of a custom molded design and universal design.

In one embodiment, the interface 18 is 2-6 millimeter surgical tubing. The interface 18 can have various shapes. In another embodiment, the dispenser 12 is 10-40 millimeters in length.

In one embodiment, the positioner 16 is inserted by the patient, removed by the patient, and/or does not require dental anchors. In other words, the patient utilizes system 10 by deploying the positioner 16 in their mouth and then removing the positioner from their mouth when their treatment is over. Stated another way, since the patient inserts and removes positioner 16, there is no need for dental anchors.

In one embodiment, the fluid is water. In another embodiment, the fluid is a fluoride treatment. In another embodiment, the fluid is an anti-bacterial solution. In another embodiment, the fluid is a water, distilled water, fluoride treatment, and/or anti-bacterial solution.

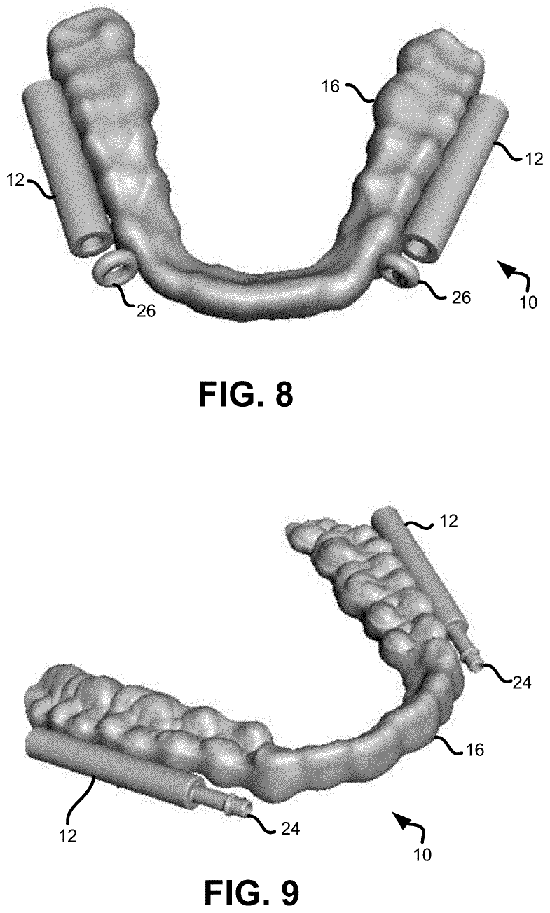

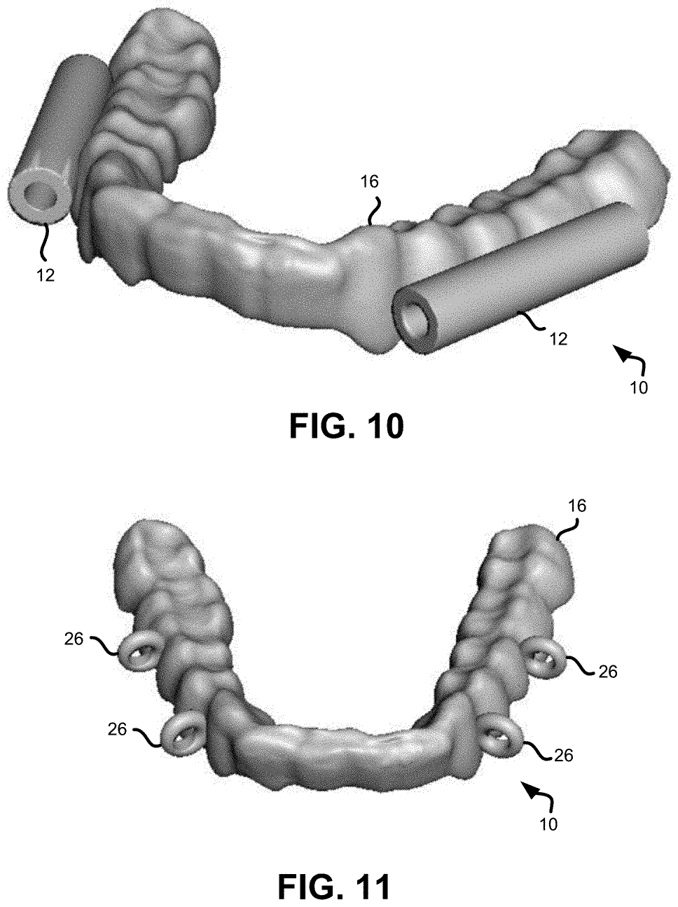

With additional reference to FIG. 8-11, in one embodiment, system 10 is a nocturnal hydration-delivery mouthpiece. In other words, the system 10 is designed to be inserted by the patient into their mouth before going to sleep, utilized while the patient sleeps, and then removed from their mouth by the patient when the patient wakes up after their sleep. In another embodiment, the system 10 is designed to be inserted by the patient into their mouth for a number of consecutive hours that are less than 24 hours and then removed from their mouth by the patient after use. In another embodiment, system 10 is paired with a pump 20 that has tubing 18 that connects it to the dispenser 12.

In one embodiment, the positioner 16 is made using either a 3D intraoral scan or a digitized polyvinyl siloxane impression to make a model. The digital model is then utilized with dental CAD software to make the “mouthpiece” part of the device.

In one embodiment, the positioner 16 can serve as a retainer and/or night guard. The positioner 16 is designed to place the patient's jaws into a therapeutic occlusion and can be in centric relation, maximum intercuspation position, etc. In another embodiment, the system 10 includes components for mandibular advancement, which will treat obstructive sleep apnea.

In one embodiment, custom designed fittings 24 are added to the sides of the dispenser 12 to accept the irrigation tubing 18. The fittings 24 are designed to accept 4 mm irrigation tubing 18, though the size of the fitting can be adjusted if the tubing changes.

In one embodiment, the tube 18 fits within the fitting 24, and in another embodiment inserts into the tubing 18. In another embodiment, rings hold the tubing 18, which would then be perforated at the ends.

In one embodiment, the dispenser 12 and/or the positioner 16 is 3D printed using a class IIa biocompatible light cured resin which is then finished in a alcohol bath for 10 minutes, then cured under UVA and UVB light for 10 minutes to ensure the resin is cured. In another embodiment other materials are used to create the dispenser 12 and/or the positioner 16.

In one embodiment, the system is designed and adjusted to allow for a therapeutic occlusal scheme, e.g. bruxism device. This allows the patient to comfortably wear the system 10 throughout the night and it will limit any harm or degradation of the patient's dentition or temporomandibular joints during the use of system 10.

In one embodiment, the dispenser 12 along the sides of the positioner 16 are perforated, e.g. port 14, using a ¼ size round dental bur to allow for delivery of the irrigating fluid. In another embodiment the port 14 is placed approximately 5-10 mm from the attachment point on the lateral dispenser 12 and are placed on the cheek facing surface spaced substantially equally.

In one embodiment, the dispenser 12 would have at least 4 ports 14 cheek facing, with holes placed on the inferior surface of the tube 18 spaced in-between the port 14. In other words, the fluid can exit openings in both the dispenser 12 and the tube 18 into the patient's mouth.

In one embodiment, the fluid only enters the patient's mouth through the dispenser 12. In another embodiment, the fluid only enters the patient's mouth through the interface 18, e.g. tube. In another embodiment, the dispenser 12 is sized on a per-patient basis and are typically sized to accommodate a 2-6 mm surgical grade tubing 18.

In one embodiment, the dispenser 12 themselves are typically 10-40 mm in length depending on the size of the dental arch being modeled to. In another embodiment, the attachment for the tubing 18 to the dispenser 12 can vary from patient to patient depending on the needs and design constraints presented by patient archforms and dentition.

In one embodiment, system 10 involves having the tubing 18 fit over a hollow protrusion, e.g. fitting 24, from the dispenser 12, which also has a raised ring to allow for an interference fit and a tight seal.

In another embodiment and with reference to FIG. 8, the system 10 utilizes rings 26 placed 2-8 mm anterior to the dispenser 12 to provide a tighter attachment. These rings are made +/−0.1 mm different on the interior diameter from the tubing 18, allowing for an interference-type fit with the flexible tubing to add retention.

In one embodiment and with additional reference to FIG. 11, is a design that utilizes the rings 26 solely to hold the medical grade tubing 18 to the positioner 16. This design is easiest to fabricate, but does not allow for ideal distribution of fluids. In one embodiment, system 10 uses fluoridated or other “medicated” irrigating liquids, e.g. fluids.

In one embodiment, the system 10 is custom made for each patient and the mechanism for attaching the irrigation tubing 18 can include a combination of the above designs depending on individual patient needs.

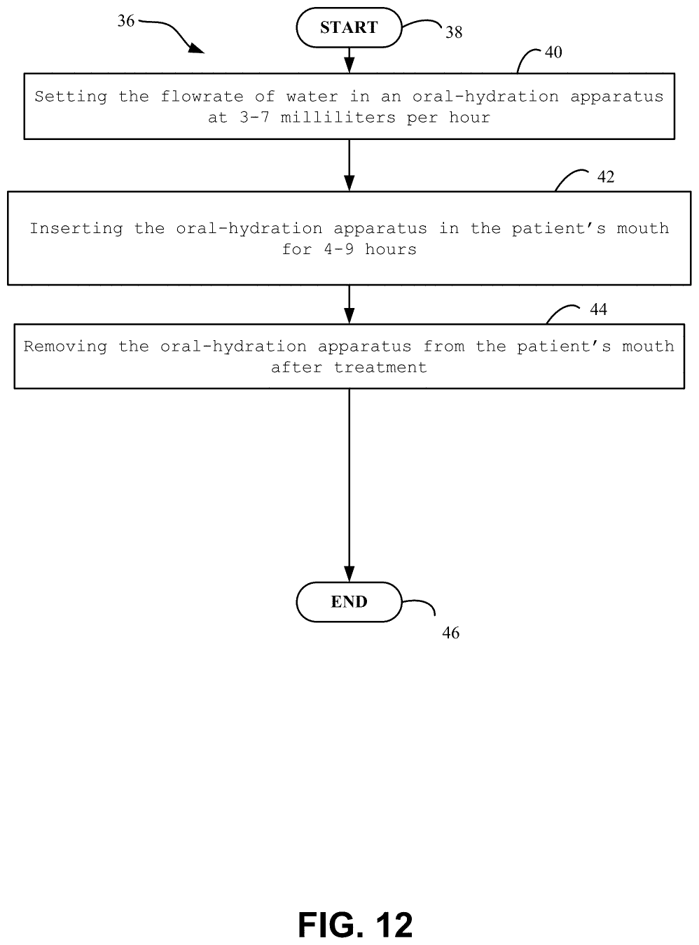

Another aspect is a method for treating a patient with water where the patient is suffering from dry mouth, which is now described with reference to flowchart 36 of FIG. 12. The method begins at Block 38 and may include setting the flowrate of water in an oral-hydration system at 3-7 milliliters per hour at Block 40. The method may also include inserting the oral-hydration system in the patient's mouth for 4-10 hours at Block 42. The method may further include removing the oral-hydration system from the patient's mouth after treatment at Block 44. The method ends at Block 46.

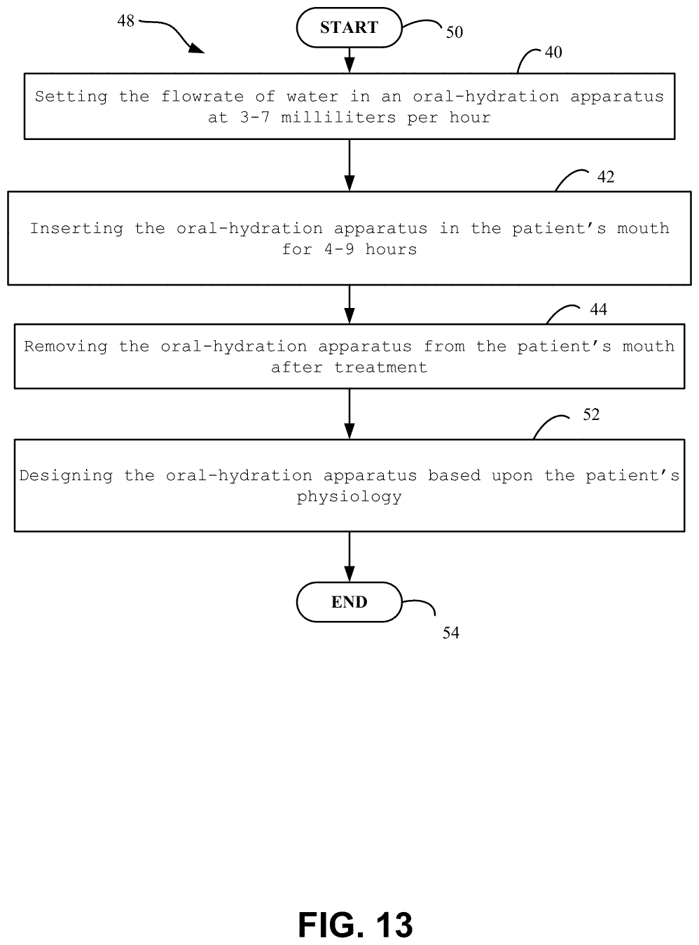

In another method of treatment embodiment, which is now described with reference to flowchart 48 of FIG. 13, the method begins at Block 50. The method may include the steps of FIG. 12 at Blocks 40-44. The method may also include designing the oral-hydration system based upon the patient's physiology at Block 52. The method ends at Block 54.

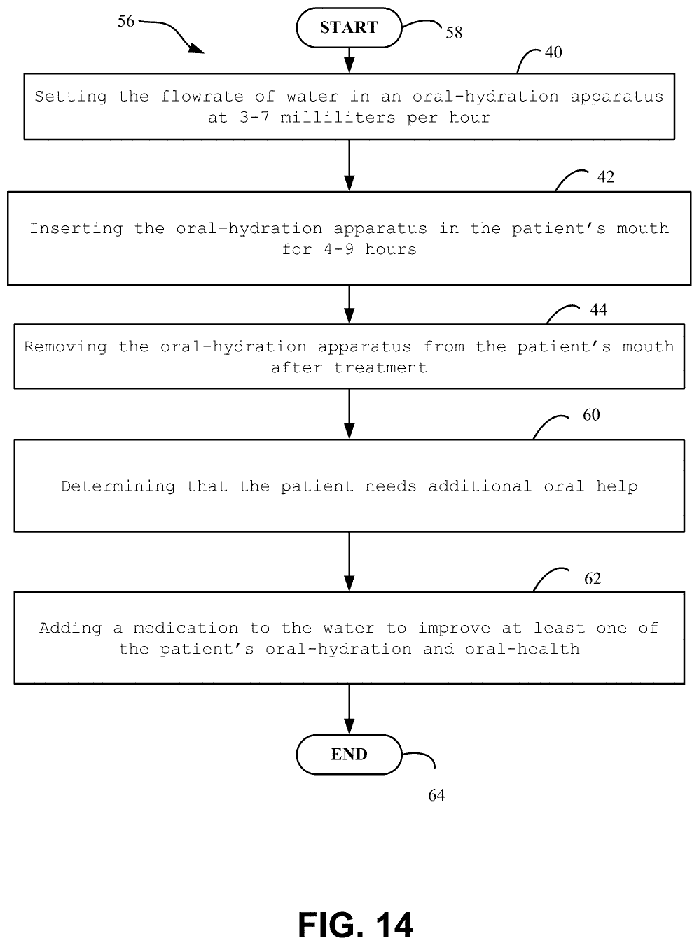

In another method of treatment embodiment, which is now described with reference to flowchart 56 of FIG. 14, the method begins at Block 58. The method may include the steps of FIG. 12 at Blocks 40-44. The method may also include determining that the patient needs additional oral help at Block 60. The method may further include adding a medication to the water to improve at least one of the patient's oral-hydration and oral-health at Block 62. The method ends at Block 64.

Another aspect is a kit for use in performing an oral-hydration procedure, and the kit includes a dispenser 12, and a port 14 carried by the dispenser and the port enables a positive-pressure fluid to be dispersed within the patient's mouth from the dispenser. The kit may further include a positioner 16 to position the dispenser 12 in contact with a premolar, adjoining a cheek, and away from an incisor of the patient. The kit may additionally include a pump 20, and an interface 18 that connects the dispenser 12 to the pump through fluid communication.

The kit may also include instructions that include dosing parameters including a flow rate through the dispenser 12, and the timing of the flow rate through the dispenser.

The kit may additionally include instructions that include setting the flowrate of the fluid in an oral-hydration system at 3-7 milliliters per hour, and inserting the oral-hydration system in the patient's mouth for 4-9 hours.

The kit may additionally include instructions to guide the patient in adapting the positioner 16 to their dental configuration. In one embodiment the instructions give fitment guidance based upon cutting the positioner 16 to size. In another embodiment, the instructions give fitment guidance to mold the positioner to the patient's dental configuration based upon heating the positioner 16 to make it softer during the molding process within the patient's own mouth. For example, the heating can be accomplished by placing the positioner 16 in heated water according to parameters outlined in the instructions. Alternatively, the heating can be accomplished by placing the positioner 16 in a warming device, e.g. oven, microwave, etc. according to parameters outlined in the instructions. Additionally, the positioner 16 comprises a thermoplastic.

Aspects of the embodiments are described above with reference to flowchart illustrations and/or block diagrams of methods, system (systems) and kit products according to the embodiments. The flowchart and block diagrams in the Figures illustrate the components, functionality, and operation of possible implementations of systems, methods and kits according to various embodiments. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the embodiments has been presented for purposes of illustration and description, but is not intended to be exhaustive or limited to the embodiments in the form disclosed. Many modifications and variations will be apparent to those of ordinary skill in the art without departing from the scope and spirit of the embodiments. The embodiment was chosen and described in order to best explain the principles of the embodiments and the practical application, and to enable others of ordinary skill in the art to understand the various embodiments with various modifications as are suited to the particular use contemplated.

While the preferred embodiment has been described, it will be understood that those skilled in the art, both now and in the future, may make various improvements and enhancements which fall within the scope of the claims which follow. These claims should be construed to maintain the proper protection for the embodiments first described.

What is claimed is:

1. An oral-hydration system comprising:
a dispenser;
a port carried by the dispenser and the port enables a fluid under positive-pressure to be dispersed within the patient's mouth from the dispenser; and
a positioner positions the dispenser adjacent a premolar, adjoining a cheek, and away from an incisor of the patient.

2. The oral-hydration system of claim 1 further comprising an interface that connects the dispenser to a pump through fluid communication.

3. The oral-hydration system of claim 2 further comprising an adjustable regulator that meters the fluid delivery to the patient.

4. The oral-hydration system of claim 1 wherein the positioner serves as at least one of a retainer, a denture, and a bruxism-guard.

5. The oral-hydration system of claim 1 wherein the port is at least one of on the cheek side of the patient's mouth, point towards the roof of the patient's mouth, and the number of ports depend on the patient's requirements.

6. The oral-hydration system of claim 1 wherein the port is at least one of on the cheek side of the patient's mouth, point towards the bottom of the patient's mouth, and the number of ports depend on the patient's requirements.

7. The oral-hydration system of claim 1 wherein the port is at least one of on the cheek side of the patient's mouth, point towards the roof and bottom of the patient's mouth, and the number of ports depend on the patient's requirements.

8. The oral-hydration system of claim 2 further comprising a fitting carried by the dispenser that joins the interface to the pump.

9. The oral-hydration system of claim 1 wherein the positioner is at least one of a custom molded design and a universal design.

10. The oral-hydration system of claim 2 wherein the interface includes 2-6 mm surgical tubing.

11. The oral-hydration system of claim 1 wherein the dispenser is 10-40 mm in length.

12. The oral-hydration system of claim 1 wherein the positioner at least one of inserted by the patient, removed by the patient, and does not require dental anchors.

13. The oral-hydration system of claim 1 wherein the fluid is at least one of water and distilled water.

14. A method for treating a patient with water, wherein the patient is suffering from dry mouth, the method comprising:
setting the flowrate of water in an oral-hydration system at 3-7 milliliters per hour;
inserting the oral-hydration system in the patient's mouth for 4-9 hours; and
removing the oral-hydration system from the patient's mouth after treatment.

15. The method of claim 12 further comprising designing the oral-hydration system based upon the patient's physiology.

16. The method of claim 12 further comprising:
determining that the patient needs additional oral help;
adding a medication to the water to improve at least one of the patient's oral-hydration and oral-health.

17. The method of claim 12 wherein the fluid is at least one of water and distilled water.

18. A kit for use in performing an oral-hydration procedure, the kit comprising:
a dispenser;
a port carried by the dispenser and the port enables a fluid under positive-pressure to be dispersed within the patient's mouth from the dispenser;
a positioner to position the dispenser in contact with a premolar, adjoining a cheek, and away from an incisor of the patient; and
a pump;
an interface that connects the dispenser to the pump through fluid communication.

19. The kit of claim 16 further comprising instructions that include dosing parameters including a flow rate through the dispenser, and the timing of the flow rate through the dispenser.

20. The kit of claim 16 further comprising instructions that include setting the flowrate of the fluid in an oral-hydration system at 3-7 milliliters per hour, and inserting the oral-hydration system in the patient's mouth for 4-9 hours.
